(19) United States
(12) Patent Application Publication
(10) Pub. No.: US 2023/0398285 A1
(43) Pub. Date: Dec. 14, 2023

(54) HEPATIC ARTERIAL INFUSION FOR TREATMENT OF LIVER AILMENTS

(71) Applicant: Medtronic, Inc., Minneapolis, MN (US)

(72) Inventor: Brian A. Duclos, Blaine, MN (US)

(21) Appl. No.: 18/144,940

(22) Filed: May 9, 2023

Related U.S. Application Data
(60) Provisional application No. 63/350,626, filed on Jun. 9, 2022.

(51) Int. Cl. A61M 5/142; A61M 5/172; A61M 25/00

(52) U.S. Cl. CPC A61M 5/142; A61M 5/172; A61M 25/0067; A61M 2205/52

(57) ABSTRACT

A system is provided for treating ailments associated with a liver of a subject. The system may comprise an implantable infusion pump or similar device that is configured to deliver a liquid formulation to the liver of the subject. In some examples, the liquid formulation may be delivered to the liver based on a catheter that is attached to the implantable infusion pump at a proximal end and implanted in a target location of the subject at a distal end, such as a hepatic artery of the subject. Additionally, the liquid formulation may be configured to be delivered at a programmable flow rate for a period of time to reach a steady state concentration in the liver for providing a treatment for an ailment associated with the liver. In some examples, the ailment associated with the liver of the patient may comprise a non-alcoholic fatty liver disease.

### CROSS-REFERENCE TO RELATED APPLICATIONS

The present application claims the benefit of and priority to U.S. Provisional Application No. 63/350,626, filed on Jun. 9, 2022, entitled “Hepatic Arterial Infusion for Treatment of Liver Ailments”, which application is incorporated herein by reference in its entirety.

### BACKGROUND

The present disclosure is generally directed to targeted drug delivery systems and, in particular, relates to delivering a therapeutic molecule to targeted areas of a subject for treating ailments associated with the liver.

Diabetes represents a large and growing global health issue with estimates of over 537 million patients worldwide having been diagnosed with type 2 diabetes and estimates of 6.7 million annual deaths related to complications of diabetes. Despite different types of treatments being developed and utilized (e.g., medication, surgery, diet, etc.), type 2 diabetes remains challenging to effectively treat. Additionally, diabetes may lead to other complications and diseases for patients. For example, type 2 diabetes mellitus (T2DM) associated non-alcoholic fatty liver disease (NAFLD) is a leading cause of death among diabetic patients. NAFLD is a spectrum of pathological conditions that include non-alcoholic steatosis (NAFL), non-alcoholic steatohepatitis (NASH), and cirrhosis.

### BRIEF SUMMARY

Example aspects of the present disclosure include:

A system for treating ailments associated with a liver of a subject, comprising: an implantable infusion pump, comprising: a reservoir configured to house a liquid formulation; an outlet in fluid communication with the reservoir; a drive mechanism configured to control a rate at which the liquid formulation is delivered to the outlet from the reservoir; and one or more electronic components operably coupled to the drive mechanism, the one or more electronic components storing data for processing, wherein the data, when processed, causes the one or more electronic components to: transmit instructions to the drive mechanism to deliver the liquid formulation from the reservoir to the outlet at a programmable flow rate for a period of time, wherein the liquid formulation is configured to be applied to the liver of the subject at the flow rate for the period of time to reach a steady state concentration in the liver for providing a treatment for an ailment associated with the liver.

Any of the aspects herein, wherein the liquid formulation comprises a therapeutic molecule comprising a parent molecule that has a molecular weight less than 5 kilodaltons (kDa), greater than 5 kDa, between about 5 kDa and about 15 kDa, between about 15 kDa and about 200 kDa, or greater than about 200 kDa.

Any of the aspects herein, wherein the parent molecule of the therapeutic molecule comprises a free base, a salt, a peptide, or another type of molecule.

Any of the aspects herein, wherein the therapeutic molecule comprises peroxisome proliferator-activated receptor (PPAR) gamma agonists; glucagon-like peptide-1 (GLP-1) agonists; dipeptidyl peptidase 4 (DPP4) inhibitors; apoptosis signal-regulating kinase 1 (ASK1) inhibitors; dual C—C motif chemokine receptor type 2 and 5 (CCR2/CCR5) antagonists; dual PPAR alpha and gamma agonists; cytokine inhibitors that include tumor necrosis factor (TNF) alpha; farnesoid X receptor (FXR) agonists; PPAR alpha agonists; steroyl-Co-A desaturase 1 (SCD1) inhibitors; pan caspase inhibitors; combined antagonists of leukotriene receptors, phosphodiesterases, and 5-lipoxygenase; galectin-3 inhibitors; 5′ adenosine monophosphate-activated protein kinase (AMPK)/Sirtuin 1 (Sirt1) pathway activators; or a combination thereof.

Any of the aspects herein, wherein the programmable flow rate comprises a flow rate of less than 500 microliters per hour.

Any of the aspects herein, further comprising: a catheter comprising a proximal end and a distal end, wherein the proximal end is coupled to the outlet of the implantable infusion pump and the distal end is configured to be implanted in a target location of the subject.

Any of the aspects herein, wherein the target location of the subject comprises a hepatic artery of the subject.

Any of the aspects herein, wherein the liquid formulation is delivered to the liver of the patient based at least in part on the distal end of the catheter being implanted in the hepatic artery of the subject.

Any of the aspects herein, wherein the ailment associated with the liver of the patient comprises a non-alcoholic fatty liver disease.

Any of the aspects herein, further comprising: an interface communicatively coupled to the one or more electronic components of the implantable infusion pump, wherein the data stored in the one or more electronic components is programmed via the interface.

A system for treating ailments associated with a liver of a subject, comprising: an implantable infusion pump; a catheter comprising a proximal end and a distal end, wherein the proximal end is coupled to the implantable infusion pump and the distal end is configured to be implanted in a target location of the subject; a processor; and a memory storing data for processing by the processor, the data, when processed, causes the processor to: transmit instructions to the implantable infusion pump to deliver a liquid formulation to the liver of the subject via the catheter based at least in part on the distal end being implanted in the target location, wherein the liquid formulation is configured to be delivered at a programmable flow rate for a period of time to reach a steady state concentration in the liver for providing a treatment for an ailment associated with the liver.

Any of the aspects herein, wherein the liquid formulation comprises a therapeutic molecule comprising a parent molecule that has a molecular weight less than 5 kDa, greater than 5 kDa, between about 5 kDa and about 15 kDa, between about 15 kDa and about 200 kDa, or greater than about 200 kDa.

Any of the aspects herein, wherein the programmable flow rate comprises a flow rate of less than 500 microliters per hour.

Any of the aspects herein, wherein the target location of the subject comprises a hepatic artery of the subject.

Any of the aspects herein, wherein the liquid formulation is delivered to the liver of the patient based at least in part on the distal end of the catheter being implanted in the hepatic artery of the subject.

Any of the aspects herein, wherein the ailment associated with the liver of the patient comprises a non-alcoholic fatty liver disease.

Any of the aspects herein, further comprising: an interface communicatively coupled to the processor, wherein the data stored in the memory is programmed via the interface.

A system for treating ailments associated with a liver of a subject, comprising: an implantable infusion pump, comprising: a reservoir configured to house a liquid formulation; an outlet in fluid communication with the reservoir; a drive mechanism configured to control a rate at which the liquid formulation is delivered to the outlet from the reservoir; and one or more electronic components operably coupled to the drive mechanism, wherein the one or more electronic components are programmed with instructions configured to cause the liquid formulation to be delivered from the reservoir to the outlet at a programmable flow rate for a period of time to reach a steady state concentration in the liver of the subject for providing a treatment for an ailment associated with the liver.

Any of the aspects herein, further comprising: a catheter comprising a proximal end and a distal end, wherein the proximal end is coupled to the outlet of the implantable infusion pump and the distal end is configured to be implanted in a hepatic artery of the subject.

Any of the aspects herein, wherein the liquid formulation is delivered to the liver of the patient based at least in part on the distal end of the catheter being implanted in the hepatic artery of the subject.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a diagram of a system according to at least one embodiment of the present disclosure;

FIG. 2 is a schematic drawing of a side view of an example infusion device system according to at least one embodiment of the present disclosure;

FIG. 3 is a flowchart according to at least one embodiment of the present disclosure; and

FIG. 4 is a block diagram of a system according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., random-access memory (RAM), read-only memory (ROM), electrically erasable programmable ROM (EEPROM), flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10× Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements. The processors listed herein are not intended to be an exhaustive list of all possible processors that can be used for implementation of the described techniques, and any future iterations of such chips, technologies, or processors may be used to implement the techniques and embodiments of the present disclosure as described herein.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator, user, or device of a system, and further from the region of medical interest in or on the patient, and distal being closer to the region of medical interest in or on the patient, and further from the operator, user, or device of the system.

Diabetes represents a large and growing global health issue with estimates of over 537 million patients worldwide having been diagnosed with type 2 diabetes and estimates of 6.7 million annual deaths related to complications of diabetes. Despite different types of treatments being developed and utilized (e.g., medication, surgery, diet, etc.), type 2 diabetes remains challenging to effectively treat. Type 2 patients must frequently contend with keeping their blood sugar levels in a desirable glycemic range. Prolonged deviations can lead to long term complications such as retinopathy, nephropathy (e.g., kidney damage), cardiovascular disease, etc. Because treatment for diabetes is self-managed by the patient on a day-to-day basis (e.g., the patients self-inject the insulin), compliance or adherence with treatments can be problematic. Additionally, in a financial sense, global expenditures for type 2 diabetes treatments, preventive measures, and resulting consequences are estimated at about $966 billion per year. Compounding this issue of high global expenditures is the increasing price of insulin.

Type 2 diabetes mellitus (T2DM) associated non-alcoholic fatty liver disease (NAFLD) is a leading cause of death among diabetic patients. NAFLD is a spectrum of pathological conditions that include non-alcoholic steatosis (NAFL), non-alcoholic steatohepatitis (NASH), and cirrhosis. A main characteristic of NAFLD is too much fat stored in liver cells. For some people, the excess fat acts as a toxin to liver cells, causing liver inflammation and NASH, which may lead to a buildup of scar tissue in the liver. For example, some individuals with NAFLD can develop NASH, an aggressive form of fatty liver disease, which is marked by liver inflammation and may progress to advanced scarring (e.g., cirrhosis) and liver failure. The damage done to the liver that is associated with NAFLD is similar to damage caused by heavy alcohol use in a patient. As the liver tries to halt inflammation, the liver may produce areas of scarring (e.g., fibrosis). With continued inflammation, fibrosis may spread to take up more and more liver tissue.

As described herein, to treat NAFLD, an enhanced drug delivery system is provided for delivering a therapeutic molecule (e.g., various therapeutics or one or more therapeutic molecules) at low doses to different cell types in a patient's liver that are responsible for NAFLD through a continuous hepatic arterial infusion (e.g., direct infusion of the therapeutic at a site of action). For example, the enhanced drug delivery system (e.g., targeted drug delivery) may include a direct to liver administration via an implantable infusion pump and catheter with a programmable flow rate to dispense a therapeutic agent to the liver utilizing a hepatic artery of the patient, such as the gastroduodenal artery or another hepatic artery. The drug delivery system and implantable infusion pump may be capable of providing a sustained, programmable drug delivery tailored to each patient for better management of various aspects of a diabetes patient's care.

The therapeutic molecule may be a therapeutic agent, a diagnostic agent, or the like. In general, a therapeutic agent is an agent intended to treat a disease, while a diagnostic agent is an agent intended to aid in identifying a condition or disease of a subject, the presence or absence of a molecule in a subject, or the like. For example, the therapeutic molecule may be any known or future developed small molecule or biologic therapeutic agent. Examples of small molecule agents that may be employed in accordance with the embodiments presented herein include, but are not limited to, peroxisome proliferator-activated receptor (PPAR) gamma agonists, such as rosiglitazone and pioglitazone as well as other thiazolidinediones; glucagon-like peptide-1 (GLP-1) agonists, such as liraglutide and exenatide; dipeptidyl peptidase 4 (DPP4) inhibitors, such as sitagliptin as well as other gliptins; apoptosis signal-regulating kinase 1 (ASK1) inhibitors, such as selonsertib; dual C—C motif chemokine receptor type 2 and 5 (CCR2/CCR5) antagonists, such as cenicriviroc; dual PPAR alpha and gamma agonists, such as elafibranor; cytokine inhibitors that include tumor necrosis factor (TNF) alpha, such as pentoxifylline; farnesoid X receptor (FXR) agonists, such as obeticholic acid; PPAR alpha agonists, such as saroglitizar; steroyl-Co-A desaturase 1 (SCD1) inhibitors, such as aramchol; pan caspase inhibitors, such as emricasan; combined antagonists of leukotriene receptors, phosphodiesterases, and 5-lipoxygenase, such as tipelukast; galectin-3 inhibitors; and 5′ adenosine monophosphate-activated protein kinase (AMPK)/Sirtuin 1 (Sirt1) pathway activators.

Generally, the therapeutic molecule(s) may be formulated into a liquid formulation suitable for delivery to the liver and/or a hepatic artery. The liquid formulation may include the therapeutic molecule(s) and a variety of other pharmaceutically acceptable components. For example, the liquid formulation may also include, depending on the formulation desired, pharmaceutically-acceptable, non-toxic carriers or diluents, which are defined as vehicles commonly used to formulate pharmaceutical compositions for animal or human administration. In most cases, the diluent is selected so as not to adversely affect the activity of the therapeutic molecule of interest. Examples of such diluents are, but are not limited to, distilled water, physiological phosphate-buffered saline, artificial cerebrospinal fluid, citrate buffered saline, Ringer's solutions, dextrose solution, and Hank's solution.

In some examples, the liquid formulations are formed as injectable compositions. Injectable compositions include solutions, suspensions, dispersions, or the like. Injectable solutions, suspensions, dispersions, or the like may be formulated using suitable dispersing or wetting and suspending agents, such as sterile oils, including synthetic mono- or diglycerides, and fatty acids, including oleic acid. Additionally, proper fluidity of solutions, suspensions, or dispersions may be maintained, for example, by the formation of liposomes, by the maintenance of the desired particle size, in the case of dispersion, or by the use of nontoxic surfactants. The prevention of microorganisms in the liquid formulations can be accomplished by heat sterilization or filter sterilization, whichever is compatible with the therapeutic molecule and liquid formulation being used. Isotonic agents such as sugars, buffers, or sodium chloride may be included. Solubility enhancers may be added.

In some embodiments, a resultant fluid composition may comprise an amount of one or more therapeutic molecules effective to treat a disease (e.g., NAFLD) to allow meaningful study of a subject to which the composition is administered at a particular flow rate (e.g., programmable flow rate). The effective amount of the therapeutic molecule to be administered may vary depending on the therapeutic molecule itself and the disease to be treated. The amount may also vary depending on the subject to which it is administered and the location of administration.

As used herein, the terms “treat” or the like means alleviating, slowing the progression, preventing, attenuating, or curing the treated disease. As used herein, “disease”, “disorder”, “condition” or the like, as they relate to a subject's health, are used interchangeably and have meanings ascribed to each and all of such terms. As used herein, “subject” means a mammal to which an agent is administered for the purposes of treatment or investigation. Mammals include mice, rats, cats, guinea pigs, hamsters, dogs, sheep, monkeys, chimpanzees, and humans. As used herein, a “liquid formulation of a therapeutic molecule” or “liquid formulation of a molecule” is a composition that contains the therapeutic molecule and that is liquid at room temperature and at a core body temperature (e.g., 37° C.).

In an exemplary embodiment, a system for treating ailments associated with the liver is provided that comprises: (i) a liquid formulation comprising a therapeutic molecule, wherein a molecular weight of a parent molecule (e.g., a free base of a salt, a peptide, or another type of molecule) of the therapeutic molecule may have any suitable molecular weight, such as less than 5 kilodaltons (kDa), greater than 5 kDa, between about 5 kDa and about 15 kDa, between about 15 kDa and about 200 kDa, or greater than about 200 kDa; and (ii) a programmable implantable infusion device comprising: (a) a reservoir configured to house the liquid formulation, (b) an outlet in fluid communication with the reservoir, (C) a drive mechanism configured to control a rate at which the liquid formulation is delivered to the outlet from the reservoir (e.g., flow rate or programmable flow rate), and (d) electronics operably coupled to the drive mechanism. In some examples, the electronics may be programmed with instructions that cause the liquid formulation to be delivered from the reservoir to the outlet at a flow rate of less than 500 microliters per hour for a period of time sufficient to reach a steady state concentration in a subject's liver if the liquid formulation is administered to a hepatic artery (e.g. gastroduodenal artery or different hepatic artery) of the subject. Accordingly, at least one attribute of an NAFLD, including NAFL, NASH, and cirrhosis, may be reduced or have its progression slowed or arrested based on delivering the liquid formulation directly to the subject's liver (e.g., via the hepatic artery).

Embodiments of the present disclosure provide technical solutions to one or more of the problems of (1) treating ailments associated with the liver (e.g., NAFLD, NAFL, NASH, cirrhosis, liver failure, etc.), (2) direct delivery of therapeutic molecule(s) to a targeted area, (3) reducing off target issues, and (4) reducing a need for patient compliance for providing treatments. For example, the embodiments, systems, and techniques described herein may include a direct infusion (e.g., via an implantable infusion device) of one or more therapeutic molecules to a subject's liver (e.g., via a hepatic artery) to treat the ailments associated with the liver. Additionally, by directly infusing the one or more therapeutic molecules to the subject's liver, a larger portion of the therapeutic molecule with smaller dosage may be delivered to the subject's liver and to cells in the liver associated with or primarily cause the ailments. The direct delivery of the therapeutic molecule(s) to the targeted area may also be performed autonomously and/or based on a set of programmed parameters, such that the subject or patient is not responsible for applying the treatment (e.g., such as the patient having to remember to take medications or self-administer treatments), thereby providing treatment for the ailments without constant or as frequent compliance from the subject or patient.

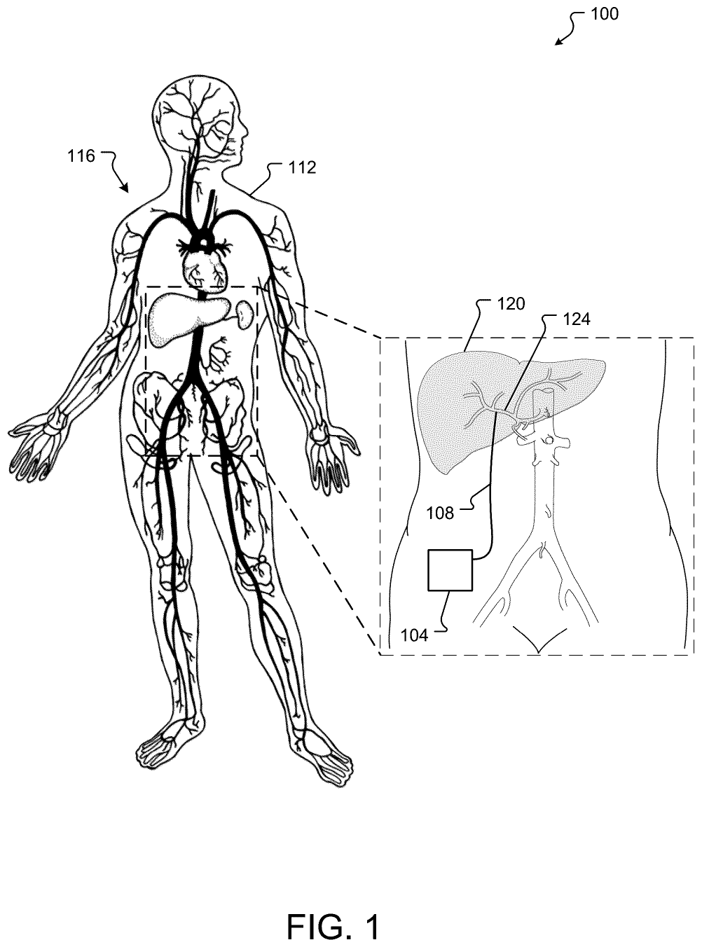

Turning to FIG. 1, a diagram of a system 100 according to at least one embodiment of the present disclosure is shown. The system 100 may be used to provide treatments to a subject or patient for ailments associated with the liver. For example, as described previously, a subject may be diagnosed with diabetes (e.g., T2DM), where the diabetes can lead to further complications, such as NAFLD, NAFL, NASH, cirrhosis, etc. Additionally or alternatively, the subject may experience ailments with their liver based on other factors that may or may not include diabetes. Accordingly, the system 100 may provide treatments for the ailments associated with the liver regardless of how the ailments were developed in the patient.

In some embodiments, the system 100 may include an implantable infusion pump 104 (e.g., which may be referred to as an infusion device, an infusion pump, a pump, a device, etc.) and a catheter 108. The implantable infusion pump 104 may be a device configured to dispense a liquid formulation at a programmable flow rate. Additionally, the implantable infusion pump 104 may be designed to be implanted below the skin of a patient 112 or subject. The implantable infusion pump 104 may be implanted in a location where the implantation interferes as little as practicable with activity of the patient 112 in which it is implanted. In some examples, the implantable infusion pump 104 may be subcutaneously implanted in the lower abdomen of the patient 112. Additionally or alternatively, the implantable infusion pump 104 may be implanted elsewhere in the patient 112. The catheter 108 may be a thin, hollow tube made from medical grade materials that is configured to transfer the liquid formulation from the implantable infusion pump 104 to a targeted area within the patient 112. The catheter 108 may be coupled to the implantable infusion pump 104 in any suitable manner (e.g., inserted into a catheter port of the implantable infusion pump 104 that is configured to accept the catheter 108).

The patient 112 may include a vascular system 116 that is made up of vessels that carry blood (and other nutrients, waste matter, lymph, etc.) through the body of the patient 112. For example, the vascular system 116 may include the heart, blood vessels (e.g., arteries, veins, capillaries, etc.), and blood. In some examples, the vascular system 116 may be referred to as the cardiovascular system of the patient 112.

As described herein, the system 100 may be used to deliver the liquid formulation from the implantable infusion pump 104 to a liver 120 of the patient 116 via the catheter 108 using one or more blood vessels of the vascular system 116. In some examples, a proximal end of the catheter 108 may be coupled to the implantable infusion pump 104 as described previously, and a distal end of the catheter 108 may be implanted in a hepatic artery 124 of the patient 112. For example, the distal end of the catheter 108 may terminate in a cylindrical hollow tube that is implanted in the hepatic artery 124 (e.g., using stereotactic surgical techniques). Accordingly, the liquid formulation may be directly delivered to the liver 120 from the implantable infusion pump 104 via the hepatic artery 124 using the catheter 108. The hepatic artery 104 may include one or more branches, such as the hepatic artery proper, gastroduodenal artery, right gastric artery, right hepatic artery, left hepatic artery, portal vein, etc. The catheter 108 may be implanted in one or more of the different branches based on the ailment being treated and/or an area or portion of the liver 120 that is being treated.

The liquid formulation stored in the implantable infusion pump 104 and delivered to the liver 120 via the hepatic artery 124 may comprise one or more therapeutic molecules that are intended to treat the ailments associated with the liver 120. For example, the therapeutic molecules (e.g., small molecule agents) may include, but are not limited to, peroxisome proliferator-activated receptor (PPAR) gamma agonists, such as rosiglitazone and pioglitazone as well as other thiazolidinediones; glucagon-like peptide-1 (GLP-1) agonists, such as liraglutide and exenatide; dipeptidyl peptidase 4 (DPP4) inhibitors, such as sitagliptin as well as other gliptins; apoptosis signal-regulating kinase 1 (ASK1) inhibitors, such as selonsertib; dual C—C motif chemokine receptor type 2 and 5 (CCR2/CCR5) antagonists, such as cenicriviroc; dual PPAR alpha and gamma agonists, such as elafibranor; cytokine inhibitors that include tumor necrosis factor (TNF) alpha, such as pentoxifylline; farnesoid X receptor (FXR) agonists, such as obeticholic acid; PPAR alpha agonists, such as saroglitizar; steroyl-Co-A desaturase 1 (SCD1) inhibitors, such as aramchol; pan caspase inhibitors, such as emricasan; combined antagonists of leukotriene receptors, phosphodiesterases, and 5-lipoxygenase, such as tipelukast; galectin-3 inhibitors; and 5′ adenosine monophosphate-activated protein kinase (AMPK)/Sirtuin 1 (Sirt1) pathway activators.

In some embodiments, the therapeutic molecule may include a parent molecule that has any suitable molecular weight, such as less than 5 kDa, greater than 5 kDa, between about 5 kDa and about 15 kDa, between about 15 kDa and about 200 kDa, or greater than about 200 kDa, where the parent molecule may comprise a free base or a salt or a peptide or another type of molecule not expressly listed herein. Additionally, the implantable infusion pump 104 may be programmable to dispense the liquid formulation at a programmable flow rate, where the programmable flow rate comprises a flow rate of less than 500 microliters per hour. Accordingly, the system 100 (e.g., drug delivery system or targeted drug delivery system) and implantable infusion pump 104 may be capable of providing a sustained, programmable drug delivery tailored to specific patients for better management of various aspects of a diabetes patient's care. Additionally, at least one attribute of an NAFLD, including NAFL, NASH, and cirrhosis, may be reduced or have its progression slowed or arrested based on the system 100 and delivering the liquid formulation directly to the liver 120 of the patient 112 (e.g., via the hepatic artery 124).

Additionally, while not shown, the system 100 may include one or more processors (e.g., one or more DSPs, general purpose microprocessors, graphics processing units, ASICs, FPGAs, or other equivalent integrated or discrete logic circuitry) that are programmed to carry out one or more aspects of the present disclosure. In some examples, the one or more processors may include a memory or may be otherwise configured to perform the aspects of the present disclosure. For example, the one or more processors may provide instructions to the implantable infusion pump 104 or other components of the system 100 not explicitly shown or described with reference to FIG. 1 for delivering the liquid formulation and therapeutic molecule(s) to the liver 120 (e.g., targeted cells in the liver 120) to provide treatments for ailments associated with the liver 120 in the patient 112 as described herein. In some examples, the one or more processors may be part of the implantable infusion pump 104 or part of a control unit for the system 100 (e.g., where the control unit is in communication with the implantable infusion pump 104 and/or other components of the system 100).

The system 100 or similar systems may be used, for example, to carry out one or more aspects of any of the methods described herein. The system 100 or similar systems may also be used for other purposes.

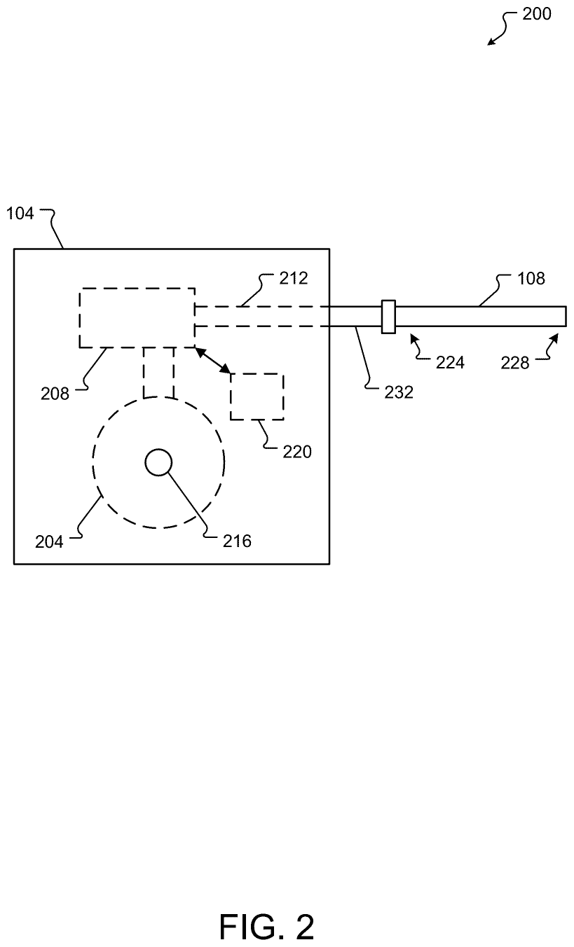

FIG. 2 depicts a schematic drawing of a side view of an example infusion device system 200 according to at least one embodiment of the present disclosure. In some examples, the infusion device system 200 may implement aspects of or may be implemented by aspects of FIG. 1 as described herein. For example, the infusion device system 200 may include the implantable infusion pump 104 as described with reference to FIG. 1. As described previously, the implantable infusion pump 104 may be used to deliver a liquid formulation comprising a therapeutic molecule directly to a liver of a subject or patient as part of treating ailments associated with the liver (e.g., NAFLD, NAFL, NASH, cirrhosis, etc.). Additionally, the infusion device system 200 may include the catheter 108 as described with reference to FIG. 1. For example, the catheter 108 may be used to transport the liquid formulation from the implantable infusion pump 104 to the liver based on being implanted or otherwise inserted into a hepatic artery of the patient.

In some embodiments, the implantable infusion pump 104 may include a reservoir 204 that is configured for housing the liquid formulation (e.g., fluid composition comprising the therapeutic molecule(s)) and a pump 208 operably coupled to the reservoir 204. In some examples, the pump 208 may include or may be referred to as a drive mechanism that is configured to control a rate at which the liquid formulation is delivered to an outlet 212 of the implantable infusion pump 104 from the reservoir 204, where the outlet 212 is in fluid communication with the reservoir 204. Additionally, the implantable infusion pump 104 may include a port 216 into which a hypodermic needle (or other medical tool) can be inserted to inject the liquid formulation into the reservoir 204. For example, the port 216 may allow for the hypodermic needle to be inserted through the skin and into the reservoir 204 to inject a quantity of a composition comprising the liquid formulation and therapeutic molecule(s) that are intended to provide treatment for the ailments associated with the liver as described herein. In some examples, the reservoir 204 may be refilled with the liquid formulation as needed using the port 216 (e.g., monthly, every 1-4 months, etc., based on a concentration of the therapeutic molecule in the liquid formulation, a frequency with which the liquid formulation is delivered to the liver, or other factors).

The implantable infusion pump 104 may be operated to discharge a predetermined dosage of the pumped fluid (e.g., liquid formulation) into a target region of a subject (e.g., into the liver of the subject, such as via a hepatic artery) at a predetermined rate (e.g., less than 500 microliters per hour). For example, the implantable infusion pump 104 may contain one or more electronic components 220 (e.g., microprocessor, processor, memory, similar electronics, etc.) that are operably coupled to the pump 208, where the one or more electronic components 220 can be programmed to control the amount and rate of fluid delivery of the pump 208. The programming may be accomplished with an external programmer/control unit via telemetry. With the use of the programmable instructions for the implantable infusion pump 104, dosage regimens of the liquid formulation may be programmed and tailored for a particular subject. Additionally, different dosages can be programmed for different combinations of fluid compositions. In some examples, the implantable infusion pump 104 may allow for starting a directed drug delivery system conservatively with lower doses and adjusting to a more aggressive dosing scheme, if warranted, based on safety and efficacy factors when used for therapeutic purposes.

The infusion device system 200 may further include the catheter 108 having a proximal end 224 coupled to the implantable infusion pump 104 and a distal end 228 configured to be implanted in a target location of a subject (e.g., a hepatic artery of the subject). Between the proximal end 224 and distal end 228 or at the distal end 228, the catheter 108 may include one or more delivery regions (not shown), such as openings through which the liquid formulation may be delivered to the subject. In some examples, the implantable infusion pump 104 may include a catheter port 232, to which the proximal end 224 of catheter 108 may be coupled. The catheter port 232 may be operably coupled to reservoir 204. As described herein, the catheter 108 may be positioned to deliver the liquid formulation and therapeutic molecule(s) to a hepatic artery, such that the liquid formulation and therapeutic molecule(s) are delivered to the liver.

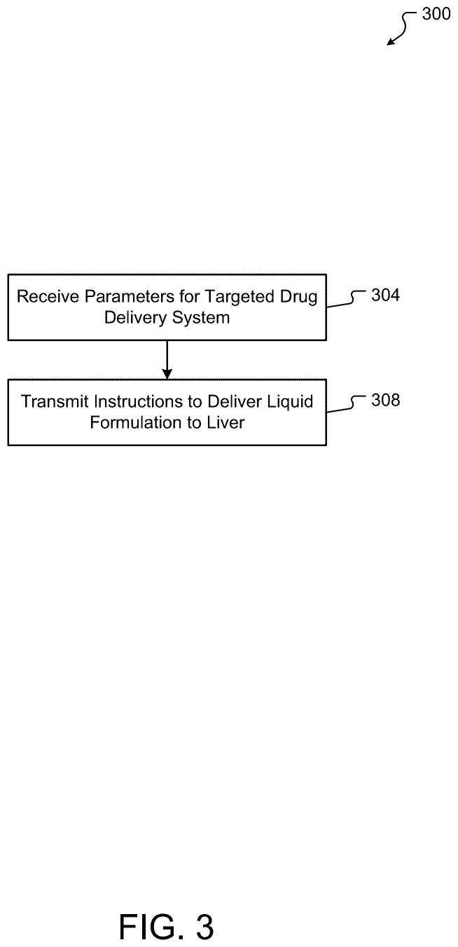

FIG. 3 depicts a method 300 that may be used, for example, to deliver a liquid formulation (e.g., comprising one or more therapeutic molecules) with a programmable flow rate to a target area within a subject for treating ailments associated with a liver of the subject.

The method 300 (and/or one or more steps thereof) may be carried out or otherwise performed, for example, by at least one processor. The at least one processor may be the same as or similar to the processor(s) of a device as described herein. The at least one processor may be part of an implantable infusion pump 104 as described with reference to FIG. 1 and/or may be part of a control unit (e.g., computing device) in communication with the implantable infusion pump 104. A processor other than any processor described herein may also be used to execute the method 300. The at least one processor may perform the method 300 by executing elements stored in a memory (such as a memory in the implantable infusion pump 104 as described herein or a control unit, computing device, etc.). The elements stored in the memory and executed by the processor may cause the processor to execute one or more steps of a function as shown in method 300. One or more portions of a method 300 may be performed by the processor executing any of the contents of memory, such as providing a targeted drug delivery for treating ailments associated with a liver of a subject and/or any associated operations as described herein.

The method 300 comprises receive parameters for a targeted drug delivery system (step 304). For example, as described herein, the targeted drug delivery system may include an implantable infusion pump and a catheter (e.g., the implantable infusion pump 104 and the catheter 108 as described with reference to FIGS. 1 and 2). The implantable infusion pump may be configured to deliver a liquid formulation to a liver of a subject via the catheter, where a proximal end of the catheter is coupled to the implantable infusion pump and a distal end of the catheter is configured to be implanted in a target location of the subject. In some examples, the target location of the subject may comprise a hepatic artery of the subject, such that the liquid formulation is delivered to the liver of the patient based on the distal end of the catheter being implanted in the hepatic artery of the subject.

In some examples, the received parameters may program one or more electronic components of the implantable infusion pump for delivering the liquid formulation to the subject. Additionally, an interface (e.g., user interface accessible by a clinician or medical professional) may be communicatively coupled to the one or more electronic components of the implantable infusion pump, such that the parameters are entered via the interface and communicated to the one or more electronic components.

In some examples, the parameters for the targeted drug delivery system may be determined to result in the liquid formulation being delivered at a programmable flow rate for a period of time to reach a steady state concentration in the liver for providing a treatment for an ailment associated with the liver. For example, the ailment associated with the liver of the patient may comprises an NAFLD (e.g., NAFL, NASH, cirrhosis, etc.). In some examples, the programmable flow rate may comprise a flow rate of less than 500 microliters per hour.

In some examples, the liquid formulation may comprise a therapeutic molecule that includes a parent molecule with any suitable molecular weight, such as less than 5 kilodaltons (kDa), greater than 5 kDa, between about 5 kDa and about 15 kDa, between about 15 kDa and about 200 kDa, or greater than about 200 kDa, where the parent molecule of the therapeutic molecule may comprise a free base or a salt or a peptide or another type of molecule. For example, the therapeutic molecule may comprise peroxisome proliferator-activated receptor (PPAR) gamma agonists; glucagon-like peptide-1 (GLP-1) agonists; dipeptidyl peptidase 4 (DPP4) inhibitors; apoptosis signal-regulating kinase 1 (ASK1) inhibitors; dual C—C motif chemokine receptor type 2 and 5 (CCR2/CCR5) antagonists; dual PPAR alpha and gamma agonists; cytokine inhibitors that include tumor necrosis factor (TNF) alpha; farnesoid X receptor (FXR) agonists; PPAR alpha agonists; steroyl-Co-A desaturase 1 (SCD1) inhibitors; pan caspase inhibitors; combined antagonists of leukotriene receptors, phosphodiesterases, and 5-lipoxygenase; galectin-3 inhibitors; 5′ adenosine monophosphate-activated protein kinase (AMPK)/Sirtuin 1 (Sirt1) pathway activators; or a combination thereof.

The method 300 also comprises transmitting instructions to the implantable infusion pump (e.g., to a drive mechanism of the implantable infusion pump) to deliver the liquid formulation (e.g., from a reservoir of the implantable infusion pump to an outlet of the implantable infusion pump) at the programmable flow rate for the period of time (step 308). In some examples, the implantable infusion pump may be configured to deliver the liquid formulation to the liver according to a programmed frequency. For example, the implantable infusion pump may deliver the liquid formulation once a day, twice a day, etc., based on the received parameters. Additionally or alternatively, the implantable infusion pump may deliver the liquid formulation on and off for different periods of time across the duration of the day to reach or maintain the steady state concentration (e.g., of the therapeutic molecule) in the liver.

The present disclosure encompasses embodiments of the method 300 that comprise more or fewer steps than those described above, and/or one or more steps that are different than the steps described above.

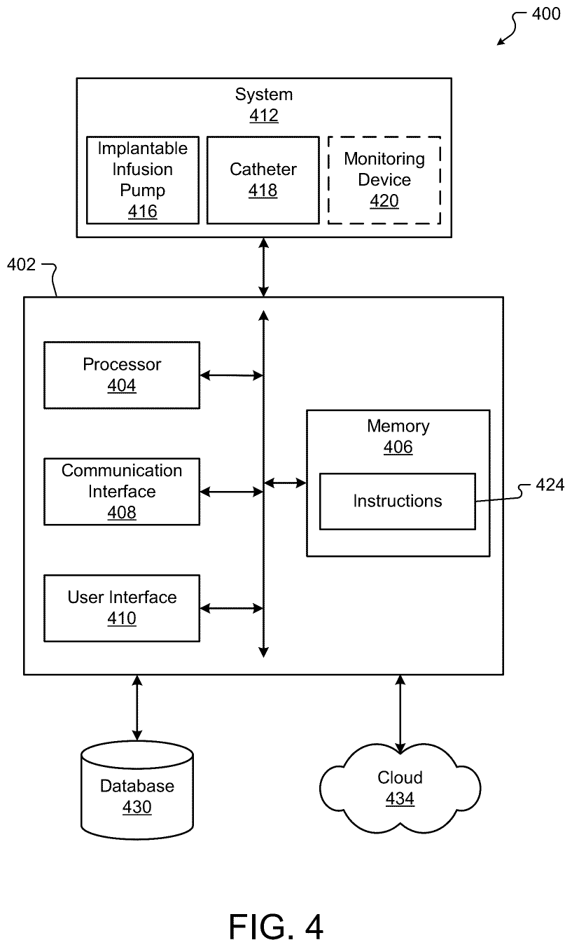

FIG. 4 depicts a block diagram of a system 400 according to at least one embodiment of the present disclosure. In some examples, the system 400 may implement aspects of or may be implemented by aspects of FIGS. 1-3 as described herein. For example, the system 400 may be used with an implantable infusion pump 416 and/or a catheter 418 and/or carry out one or more other aspects of one or more of the methods disclosed herein. The implantable infusion pump 416 may represent an example of the implantable infusion pump 104 or a component of the implantable infusion pump 104 as described with reference to FIGS. 1 and 2, and the catheter 418 may represent an example of the catheter 108 as described with reference to FIGS. 1 and 2. Additionally or alternatively, the system 400 may be used with a monitoring device 420 and/or may carry out one or more other aspects of one or more of the methods disclosed herein. The system 400 comprises a computing device 402, a system 412, a database 430, and/or a cloud or other network 434. Systems according to other embodiments of the present disclosure may comprise more or fewer components than the system 400. For example, the system 400 may not include one or more components of the computing device 402, the database 430, and/or the cloud 434.

The system 412 may comprise the implantable infusion pump 416 and the catheter 418. As previously described, the implantable infusion pump 416 may configured to deliver a liquid formulation comprising a therapeutic molecule directly to a liver of a subject or patient as part of treating ailments associated with the liver (e.g., NAFLD, NAFL, NASH, cirrhosis, etc.). Additionally, the catheter 418 may be configured to transport the liquid formulation from the implantable infusion pump 416 to the liver based on being implanted or otherwise inserted into a hepatic artery of the patient. The system 412 may communicate with the computing device 402 to receive instructions such as instructions 424 for programming the implantable infusion pump 416 to deliver the liquid formulation at a programmable flow rate (e.g., less than 500 microliters per hour), at a programmable frequency, and/or other parameters for delivering the liquid formulation to the liver of the patient. In some examples, the instructions 424 may be entered or otherwise provided by a clinician or medical professional that has determined the programmable parameters specific to a given patient to efficaciously provide the treatments for the ailments associated with the liver of the given patient.

The system 412 may also provide data (such as data received from a monitoring device 420 capable of recording data), which may be used to optimize the programmable flow rate and/or other parameters for delivering the liquid formulation to the liver of the patient via the implantable infusion pump 416. For example, the monitoring device 420 may monitor a concentration of the liquid formulation and/or therapeutic molecule(s) in the liver of the patient to determine when the implantable infusion pump 416 is to start or stop delivery of the liquid formulation, increase or decrease the flow rate, etc. Additionally or alternatively, the instructions 424 may statically configure the various parameters with which the liquid formulation and/or therapeutic molecule(s) are delivered to the liver until the instructions 424 are updated or adjusted (e.g., by the clinician or medical professional in a clinical setting).

The computing device 402 comprises a processor 404, a memory 406, a communication interface 408, and a user interface 410. Computing devices according to other embodiments of the present disclosure may comprise more or fewer components than the computing device 402.

The processor 404 of the computing device 402 may be any processor described herein or any similar processor. The processor 404 may be configured to execute instructions 424 stored in the memory 406, which instructions may cause the processor 404 to carry out one or more computing steps utilizing or based on data received from the system 412, the database 430, and/or the cloud 434.

The memory 406 may be or comprise RAM, DRAM, SDRAM, other solid-state memory, any memory described herein, or any other tangible, non-transitory memory for storing computer-readable data and/or instructions. The memory 406 may store information or data useful for completing, for example, any steps of the methods 300 and/or 400 described herein, or of any other methods. The memory 406 may store, for example, instructions and/or machine learning models that support one or more functions of the system 412. For instance, the memory 406 may store content (e.g., instructions 424 and/or machine learning models) that, when executed by the processor 404, cause the implantable infusion pump 416 to deliver the liquid formulation comprising the therapeutic molecule directly to the liver of the subject or patient as part of treating ailments associated with the liver.

Content stored in the memory 406, if provided as in instruction, may, in some embodiments, be organized into one or more applications, modules, packages, layers, or engines. Alternatively or additionally, the memory 406 may store other types of content or data (e.g., machine learning models, artificial neural networks, deep neural networks, etc.) that can be processed by the processor 404 to carry out the various method and features described herein. Thus, although various contents of memory 406 may be described as instructions, it should be appreciated that functionality described herein can be achieved through use of instructions, algorithms, and/or machine learning models. The data, algorithms, and/or instructions may cause the processor 404 to manipulate data stored in the memory 406 and/or received from or via the system 412, the database 430, and/or the cloud 434.

The computing device 402 may also comprise a communication interface 408. The communication interface 408 may be used for receiving data (for example, data from the monitoring device 420) or other information from an external source (such as the system 412, the database 430, the cloud 434, and/or any other system or component not part of the system 400), and/or for transmitting instructions, images, or other information to an external system or device (e.g., another computing device 402, the system 412, the database 430, the cloud 434, and/or any other system or component not part of the system 400). The communication interface 408 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 408 may be useful for enabling the device 402 to communicate with one or more other processors 404 or computing devices 402, whether to reduce the time needed to accomplish a computing-intensive task or for any other reason.

The computing device 402 may also comprise one or more user interfaces 410. The user interface 410 may be or comprise a keyboard, mouse, trackball, monitor, television, screen, touchscreen, and/or any other device for receiving information from a user and/or for providing information to a user. The user interface 410 may be used, for example, to receive a user selection or other user input regarding any step of any method described herein. Notwithstanding the foregoing, any required input for any step of any method described herein may be generated automatically by the system 400 (e.g., by the processor 404 or another component of the system 400) or received by the system 400 from a source external to the system 400. In some embodiments, the user interface 410 may be useful to allow a surgeon or other user to modify instructions to be executed by the processor 404 according to one or more embodiments of the present disclosure, and/or to modify or adjust a setting of other information displayed on the user interface 410 or corresponding thereto. For example, the user interface 410 may be communicatively coupled to the implantable infusion pump 416 (e.g., to one or more electronic components of the implantable infusion pump 416 as described herein), where data and/or the instructions 424 stored in the implantable infusion pump 416 for programming the parameters with which the liquid formulation (e.g., programmed by a clinician or other medical professional) is delivered from the implantable infusion pump 416 to the liver is programmed via the user interface 410.

Although the user interface 410 is shown as part of the computing device 402, in some embodiments, the computing device 402 may utilize a user interface 410 that is housed separately from one or more remaining components of the computing device 402. In some embodiments, the user interface 410 may be located proximate one or more other components of the computing device 402, while in other embodiments, the user interface 410 may be located remotely from one or more other components of the computer device 402.

Though not shown, the system 400 may include a controller, though in some embodiments the system 400 may not include the controller. The controller may be an electronic, a mechanical, or an electro-mechanical controller. The controller may comprise or may be any processor described herein. The controller may comprise a memory storing instructions for executing any of the functions or methods described herein as being carried out by the controller. In some embodiments, the controller may be configured to simply convert signals received from the computing device 402 (e.g., via a communication interface 408) into commands for operating the system 412 (and more specifically, for actuating the implantable infusion pump 416). In other embodiments, the controller may be configured to process and/or convert signals received from the system 412. Further, the controller may receive signals from one or more sources (e.g., the system 412) and may output signals to one or more sources.

The database 430 may store information such as patient data, flow parameters, etc. The database 430 may be configured to provide any such information to the computing device 402 or to any other device of the system 400 or external to the system 400, whether directly or via the cloud 434. In some embodiments, the database 430 may be or comprise part of a hospital image storage system, such as a picture archiving and communication system (PACS), a health information system (HIS), and/or another system for collecting, storing, managing, and/or transmitting electronic medical records.

The cloud 434 may be or represent the Internet or any other wide area network. The computing device 402 may be connected to the cloud 434 via the communication interface 408, using a wired connection, a wireless connection, or both. In some embodiments, the computing device 402 may communicate with the database 430 and/or an external device (e.g., a computing device) via the cloud 434.

The system 400 or similar systems may be used, for example, to carry out one or more aspects of any of the method 300 as described herein. The system 400 or similar systems may also be used for other purposes.

As noted above, the present disclosure encompasses methods with fewer than all of the steps identified in FIG. 3 (and the corresponding description of the method 300), as well as methods that include additional steps beyond those identified in FIG. 3 (and the corresponding description of the method 300). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for treating ailments associated with a liver of a subject, comprising:
an implantable infusion pump, comprising:
a reservoir configured to house a liquid formulation;
an outlet in fluid communication with the reservoir;
a drive mechanism configured to control a rate at which the liquid formulation is delivered to the outlet from the reservoir; and
one or more electronic components operably coupled to the drive mechanism, the one or more electronic components storing data for processing, wherein the data, when processed, causes the one or more electronic components to:
transmit instructions to the drive mechanism to deliver the liquid formulation from the reservoir to the outlet at a programmable flow rate for a period of time, wherein the liquid formulation is configured to be applied to the liver of the subject at the flow rate for the period of time to reach a steady state concentration in the liver for providing a treatment for an ailment associated with the liver.

2. The system of claim 1, wherein the liquid formulation comprises a therapeutic molecule comprising a parent molecule that has a molecular weight less than 5 kilodaltons (kDa), greater than 5 kDa, between about 5 kDa and about 15 kDa, between about 15 kDa and about 200 kDa, or greater than about 200 kDa.

3. The system of claim 2, wherein the parent molecule of the therapeutic molecule comprises a free base, a salt, a peptide, or another type of molecule.

4. The system of claim 2, wherein the therapeutic molecule comprises peroxisome proliferator-activated receptor (PPAR) gamma agonists; glucagon-like peptide-1 (GLP-1) agonists; dipeptidyl peptidase 4 (DPP4) inhibitors; apoptosis signal-regulating kinase 1 (ASK1) inhibitors; dual C—C motif chemokine receptor type 2 and 5 (CCR2/CCR5) antagonists; dual PPAR alpha and gamma agonists; cytokine inhibitors that include tumor necrosis factor (TNF) alpha; farnesoid X receptor (FXR) agonists; PPAR alpha agonists; steroyl-Co-A desaturase 1 (SCD1) inhibitors; pan caspase inhibitors; combined antagonists of leukotriene receptors, phosphodiesterases, and 5-lipoxygenase; galectin-3 inhibitors; 5′ adenosine monophosphate-activated protein kinase (AMPK)/Sirtuin 1 (Sirt1) pathway activators; or a combination thereof.

5. The system of claim 1, wherein the programmable flow rate comprises a flow rate of less than 500 microliters per hour.

6. The system of claim 1, further comprising:
a catheter comprising a proximal end and a distal end, wherein the proximal end is coupled to the outlet of the implantable infusion pump and the distal end is configured to be implanted in a target location of the subject.

7. The system of claim 6, wherein the target location of the subject comprises a hepatic artery of the subject.

8. The system of claim 7, wherein the liquid formulation is delivered to the liver of the patient based at least in part on the distal end of the catheter being implanted in the hepatic artery of the subject.

9. The system of claim 1, wherein the ailment associated with the liver of the patient comprises a non-alcoholic fatty liver disease.

10. The system of claim 1, further comprising:
an interface communicatively coupled to the one or more electronic components of the implantable infusion pump, wherein the data stored in the one or more electronic components is programmed via the interface.

11. A system for treating ailments associated with a liver of a subject, comprising:
an implantable infusion pump;
a catheter comprising a proximal end and a distal end, wherein the proximal end is coupled to the implantable infusion pump and the distal end is configured to be implanted in a target location of the subject;
a processor; and
a memory storing data for processing by the processor, the data, when processed, causes the processor to:
transmit instructions to the implantable infusion pump to deliver a liquid formulation to the liver of the subject via the catheter based at least in part on the distal end being implanted in the target location, wherein the liquid formulation is configured to be delivered at a programmable flow rate for a period of time to reach a steady state concentration in the liver for providing a treatment for an ailment associated with the liver.

12. The system of claim 11, wherein the liquid formulation comprises a therapeutic molecule comprising a parent molecule that has a molecular weight less than 5 kilodaltons (kDa), greater than 5 kDa, between about 5 kDa and about 15 kDa, between about 15 kDa and about 200 kDa, or greater than about 200 kDa.

13. The system of claim 11, wherein the programmable flow rate comprises a flow rate of less than 500 microliters per hour.

14. The system of claim 11, wherein the target location of the subject comprises a hepatic artery of the subject.

15. The system of claim 14, wherein the liquid formulation is delivered to the liver of the patient based at least in part on the distal end of the catheter being implanted in the hepatic artery of the subject.

16. The system of claim 11, wherein the ailment associated with the liver of the patient comprises a non-alcoholic fatty liver disease.

17. The system of claim 11, further comprising:
an interface communicatively coupled to the processor, wherein the data stored in the memory is programmed via the interface.

18. A system for treating ailments associated with a liver of a subject, comprising:
an implantable infusion pump, comprising:
a reservoir configured to house a liquid formulation;
an outlet in fluid communication with the reservoir;
a drive mechanism configured to control a rate at which the liquid formulation is delivered to the outlet from the reservoir; and
one or more electronic components operably coupled to the drive mechanism, wherein the one or more electronic components are programmed with instructions configured to cause the liquid formulation to be delivered from the reservoir to the outlet at a programmable flow rate for a period of time to reach a steady state concentration in the liver of the subject for providing a treatment for an ailment associated with the liver.

19. The system of claim 18, further comprising:
a catheter comprising a proximal end and a distal end, wherein the proximal end is coupled to the outlet of the implantable infusion pump and the distal end is configured to be implanted in a hepatic artery of the subject.

20. The system of claim 19, wherein the liquid formulation is delivered to the liver of the patient based at least in part on the distal end of the catheter being implanted in the hepatic artery of the subject.
